(19) United States
(12) Patent Application Publication
(10) Pub. No.: US 2024/0181228 A1
(43) Pub. Date: Jun. 6, 2024

(54) SYSTEM FOR PREPARING THE GENITAL TRACT OF A WOMAN BEFORE A CHILDBIRTH

(71) Applicant: MUMMING, Bègles Cedex (FR)

(72) Inventors: Thomas ZUBER, Bordeaux (FR); Gonzague MELLERIO, Paris (FR)

(21) Appl. No.: 18/285,984

(22) PCT Filed: Apr. 6, 2022
(86) PCT No.: PCT/FR2022/050642

§ 371 Date: Oct. 6, 2023

(30) Foreign Application Priority Data
Apr. 7, 2021 (FR) FR2103560

(51) Int. Cl. A61M 29/02; A61M 25/10

(52) U.S. Cl. CPC A61M 29/02; A61M 25/10188; A61M 2205/3306; A61M 2205/3327; A61M 2205/3375; A61M 2205/3592; A61M 2205/586; A61M 2210/1475

(57) ABSTRACT

A system for preparing the genital tract of a woman prior to childbirth, the system including:—an inflatable body intended to be placed in the genital tract of the woman, the inflatable body comprising a wall having a first, open end intended to allow air to enter the inflatable body or leave the inflatable body, and a second, closed end opposite the first end,—an inflating device serving to inflate the inflatable body and connected fluidically to the first, open end of the inflatable body by a conduit;—a measuring device configured to measure a dimension of the inflatable body,—a control device configured to collect the measurement data from the measuring device.

### CROSS REFERENCE TO RELATED APPLICATIONS

This application is a National Stage of PCT Application No. PCT/FR2022/050642 filed on Apr. 6, 2022, which claims priority to French Patent Application No. FR 21/03560 filed on Apr. 7, 2021, the entire contents each of which are incorporated herein by reference thereto.

### TECHNICAL FIELD

The present disclosure relates to a system intended to prepare the genital tract of a woman before a childbirth. In particular, the present disclosure relates to a connected system comprising an inflatable body intended to be placed in the genital tract of the woman with a view to softening the genital tract, a measuring device configured to measure a dimension of the inflatable body and whose dimension measurement data is collected in real time to allow the woman to follow her progress.

### BACKGROUND

During a natural childbirth, the genital tract (comprising the vagina, pelvic floor and perineum) of a woman is subjected to a stretching which can cause unwanted damage.

In certain cases, an episiotomy is performed to facilitate the passage of the baby.

This surgical procedure can have significant consequences for the woman such as more or less intense physical and psychological pain and can also require a certain amount of healing time.

To overcome these drawbacks, there are childbirth preparation exercises which will make it possible to prepare the genital tract for the passage of the baby during the childbirth.

There are different types of system allowing to carry out these exercises. It is known, for example, the system known as Epi-no® which comprises an inflatable body intended to be placed in the genital tract of a user and which is configured to be inflated manually by means of an inflation bulb connected to the inflatable body through a conduit. In operation, the user inflates the inflatable body using the inflation bulb. The inflatable body exerts a force on the muscles of the user's genital tract and allows a progressive softening of the user's genital tract. The expansion of the inflatable body can gradually be intensified during the preparation sessions.

A drawback of this system is that it is not possible to reliably quantify the expansion of the inflatable body and therefore to be able to precisely follow the evolution of the softening of the genital tract.

Another drawback of this system is that it is not possible for the user to obtain information relating to the progress of her preparation in real time and in a simple manner.

### BRIEF SUMMARY

The present disclosure aims to resolve all or part of the drawbacks mentioned above.

This presentation concerns a system for preparing the genital tract of a woman before the childbirth, the system comprising:
an inflatable body intended to be placed in the female genital tract, the inflatable body comprising a wall including a first open end intended to allow air to enter the inflatable body or air to leave the inflatable body and a second closed end opposite the first end,
a device for inflating the inflatable body fluidly connected to the first open end of the inflatable body by a conduit.
a measuring device configured to measure a dimension of the inflatable body,
a control device configured to collect the dimension measurement data of the measuring device.

The system according to the present disclosure allows muscle, skin and mucous membrane preparation for the childbirth to avoid lesions and their consequences.

The system according to the present presentation makes it possible to prepare the genital tract of the woman in order to facilitate the childbirth.

The present disclosure further allows real-time monitoring of the evolution by the user and better control of her perineum.

According to one aspect, the wall of the inflatable body has a tubular portion of constant or variable section.

According to one embodiment, the control device is arranged to transmit the dimension measurement data of the measuring device to an interface device configured to receive and display dimension measurement data of the measuring device, in particular through a wireless communication link.

According to one embodiment, the interface device comprises a computer application executed on a mobile communications terminal. The system may comprise the interface device. Alternatively, the interface device may consist of an application or a third-party device arranged to process the dimension measurement data of the measuring device.

The present disclosure makes it possible to provide a system in which the dimensions of the inflatable body are known in real time and transmitted to the user via, for example, an object having a screen (telephone, tablet, computer, etc.).

An advantage of the present disclosure lies in the ability to read and transcribe in real time the use data of the inflatable body in order to guide the user towards proper use of the device and thus improve their progress.

According to one embodiment, the measuring device is configured to measure a dimension of the inflatable body corresponding to a cross section of the inflatable body.

By transverse section is meant a section of the inflatable body considered transversely to a direction of insertion of the inflatable body into the genital tract.

According to one embodiment, the inflatable body has a substantially circular cross-section when the body is inflated. In this embodiment, the system is arranged to determine a diameter of said cross section of the inflatable body.

According to one embodiment, the measuring device is integrated into the inflatable body and comprises at least one distance sensor. In this embodiment, the inflatable body comprises a central support extending into the inflatable body and the distance sensor is arranged on the central support of the inflatable body, the measuring device being configured to measure a distance between the central support and a point located on an inner face of the wall of the inflatable body.

According to one characteristic, the central support is a semi-rigid rod and said at least one sensor is arranged on a free end of the semi-rigid rod.

According to one characteristic, said at least one distance sensor is a sensor selected from the group consisting of an infrared sensor, an ultrasonic sensor and a laser sensor.

According to one characteristic, said at least one distance sensor is a shooting device and the inflatable body comprises at least one pattern or a rib arranged on an inner face of the wall of the inflatable body.

For example, the shooting device is a camera, a digital camera.

According to one embodiment, the system comprises a first part intended to be placed in the genital tracts and a second part intended to remain outside the genital tracts. The first part comprises the inflatable body and the second part comprises the inflation device, the control device and the measuring device, the measuring device comprising at least one pressure sensor and at least one computer configured to calculate a quantity of air entering the inflatable body and/or leaving the inflatable body.

According to one characteristic, the device comprises at least one temperature sensor.

Thus, the measuring device is arranged to determine the dimension of the inflatable body as a function of a pressure value, a temperature value and a quantity of air in the inflatable body. It should be noted that the measuring device may comprise for this purpose a processing unit which may be distinct or shared with the control device and/or with the measuring device.

According to one characteristic, the computer is configured to calculate a quantity of air entering the inflatable body using information communicated by a motor.

According to one characteristic, the computer is configured to calculate a quantity of air leaving the inflatable body using information communicated by a solenoid valve.

According to one characteristic, the second end of the inflatable body comprises a bulge.

According to one characteristic, the system comprises gripping means intended to allow a positioning of the inflatable body in the woman's genital tract, the gripping means being a semi-rigid handle.

According to one characteristic, the semi-rigid handle is telescopic.

Other characteristics and advantages of the present disclosure will appear on reading the non-limiting description which follows and the appended figures which schematically illustrate several embodiments of the system according to the present disclosure.

### BRIEF DESCRIPTION OF THE DRAWINGS

FIG. 1 is an illustration of a childbirth preparation system according to an embodiment of the present disclosure.

FIG. 2 is an enlarged view of an inflatable body of the preparation system according to the present disclosure.

FIG. 3 illustrates the system comprising a measuring device according to a first embodiment of the present disclosure.

FIG. 4 illustrates a variant of the measuring device according to the first embodiment.

FIG. 5 illustrates the system comprising a measuring device according to a second embodiment of the present disclosure.

FIG. 6 illustrates the inflatable body of the system according to a variant.

FIG. 7 illustrates the inflatable body of the system according to a variant.

FIG. 8 illustrates the inflatable body of the system according to a variant.

FIG. 9 illustrates the inflatable body of the system according to a variant.

FIG. 10 illustrates the inflatable body of the system according to a variant.

FIG. 11 illustrates the inflatable body of the system according to a variant.

FIG. 12 illustrates the inflatable body of the system according to a variant.

### DETAILED DESCRIPTION

For the sake of simplification, identical elements are identified by identical reference signs in all of the figures.

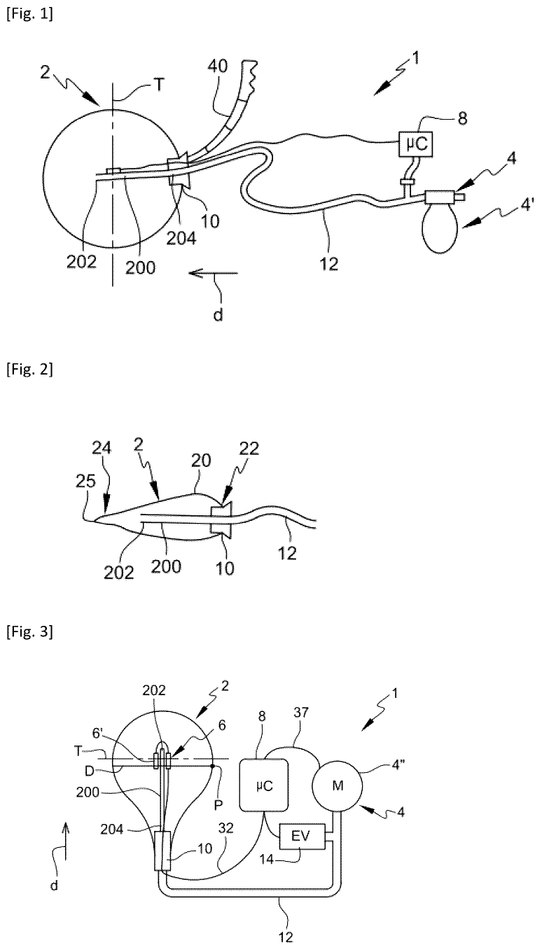

FIG. 1 illustrates a system 1 for preparing a woman for childbirth according to the present presentation.

The system 1 comprises an inflatable body 2 intended to be placed in the woman's genital tract, an inflation device 4 intended for inflating the inflatable body, a measuring device 6 configured to measure a dimension of the inflatable body and a control device 8 configured to collect dimension measurement data from measuring device 6.

The inflatable body 2 is configured to exert a force on the muscles of the female genital tract with the aim of softening the genital tract when the body is inflated, for example by means of a gas such as air.

The inflatable body 2 comprises a material compatible with the human body, for example the inflatable body 2 can comprise latex or silicone.

As illustrated in FIG. 2, the inflatable body 2 comprises a wall 20 including a first open end 22 intended to allow air to enter the inflatable body or air to exit from the inflatable body and a second closed end 24 opposite the first end 22.

According to one aspect, the wall of the inflatable body has a portion of constant or variable section.

For example, the wall of the inflatable body has a tubular portion of constant or variable section.

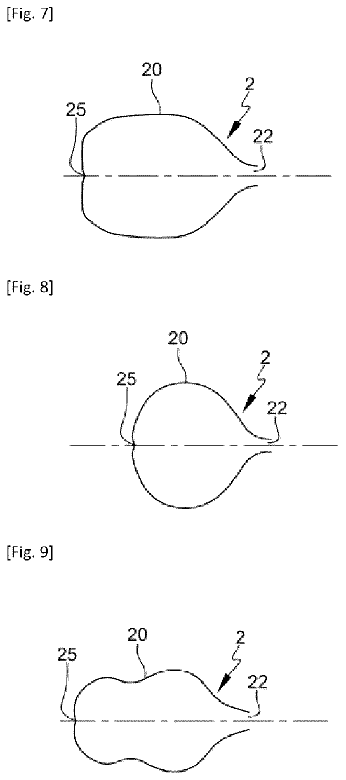

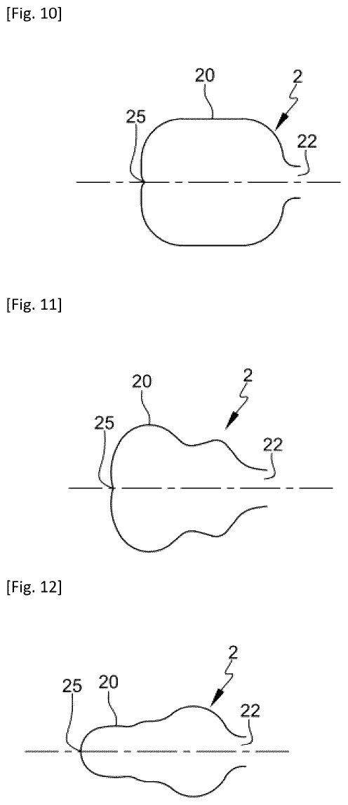

Examples of shapes of the wall are shown in FIGS. 6 to 12 which illustrate longitudinal sectional views of the inflatable body according to several embodiments.

In the shown example, the second end 24 may comprise a bulge 25. The bulge 25 allows a lubricated element to be attached to the inflatable body to allow facilitating an insertion of the inflatable body into the genital tract of the user.

The inflatable body 2 may comprise a central support 200 extending into the inflatable body 2. For example, the central support 200 is a semi-rigid rod which makes it possible to facilitate the insertion of the inflatable body 2 into the genital tract of the woman. The semi-rigid rod comprises a first free end 202 extending at a median section of the inflatable body 2 and a second end 204 connected to the open end 22 of the inflatable body 2. For example, the semi-rigid rod is fixed on a collar forming a handle 10 on which the inflatable body 2 is fixed. The handle 10 also makes it possible to guide the insertion of the inflatable body 2 into the genital tract.

When the body is inflated, the body has a substantially circular «T» cross section as shown in FIGS. 1, 3, 4 and 5.

By cross section «T» is meant a section of the inflatable body considered transversely to a direction of insertion «d» of the inflatable body in the genital tract. In this configuration, the inflatable body also makes it possible to simulate a passage of a baby's head.

The inflatable body has a determined thickness so that the inflatable body does not deflate even if the user exerts a force on the inflatable body. The system thus allows the user to exercise these muscles by applying pressure on the inflatable body during an exercise for example.

The inflatable body 2 is inflated by means of the inflation device 4 which is fluidly connected to the open end 204 of the inflatable body 2 by a conduit 12 such as a flexible pipe.

According to a first embodiment illustrated in FIG. 1, the inflation device 4 comprises a manual inflation bulb 4′. In this embodiment, the manual bulb may comprise a valve (not shown) allowing the inflatable body 2 to be slightly or completely deflated.

According to a second embodiment illustrated in FIGS. 3 to 5, the inflation device 4 is a motorized device and comprises a motor 4″.

The motor operates like an air pump.

For example, the motor is a solenoid electric motor including an adapter intended to pump air from the atmosphere via an opening provided for this purpose.

In this second embodiment, the system comprises a solenoid valve 14 making it possible to deflate the inflatable body 2 slightly or completely. The motor 4″ and the solenoid valve 12 are connected to the control device by conductive wires 36, 37.

The measuring device 6 is configured to measure a dimension of the inflatable body during inflation of the body. In particular, the measuring device is configured to measure a dimension of the body corresponding to the cross section «T» of the inflatable body 2.

Furthermore, the measuring device 6 allows the system 1 to be arranged to determine a diameter «D» of the circular cross section of the inflatable body as a function of the measurements measured by the measuring device 6.

The dimension measurement data of the measuring device 6 are further collected by the control device 8.

The control device 8 comprises for example a microcontroller. The system comprises a battery (not shown) intended to power the microcontroller.

The control device 8 is arranged to transmit the dimension measurement data from the measuring device to an interface device (not shown) configured to receive and display dimension measurement data from the measuring device, in particular by a wireless communications link.

According to one embodiment, the interface device comprises a computer application executed on a mobile communications terminal.

The system may comprise the interface device.

Alternatively, the interface device may consist of an application or a third-party device arranged to process the dimension measurement data of the measuring device.

The system makes it possible to display the information to the user in real time and can also compare it instantaneously with measurements usually observed.

In order to provide even more relevant information, the system can be arranged to allow the user to enter information about her pregnancy (number of weeks of amenorrhea, age, weight, height, shoe size). If the user has integrated this data into the system, the system will then compare the measurements collected in real time with those usually observed for a person of the same age, of the same size and at the same time of their pregnancy.

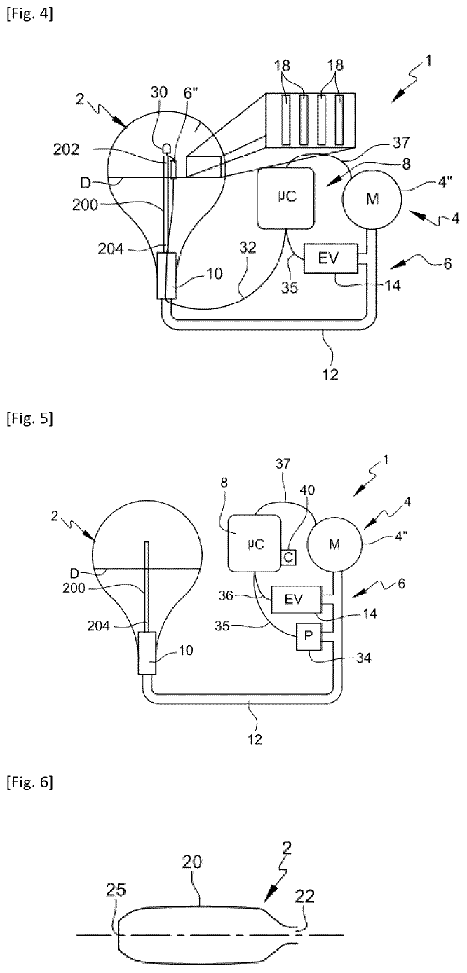

According to a first embodiment illustrated in FIGS. 3 and 4, the measuring device 6 is integrated into the inflatable body 2.

In this embodiment, the measuring device comprises at least one distance sensor 6′.

In the shown examples, a sensor is arranged on the central support 200 of the inflatable body 2.

For example, when the central support 200 is a semi-rigid rod, the distance sensor 6′ is arranged on the free end 202 of the rod of the inflatable body.

The measuring device 6 is configured to measure a distance between the central support 200 and a point «P» located on an inner face of the wall 20 of the inflatable body.

Furthermore, this distance corresponds to a radius of the inflatable body.

For example, the distance sensor 6′ is an infrared sensor.

The infrared sensor comprises a transmitter and a receiver. The transmitter is configured to emit infrared light toward the wall of the inflatable body that is returned by the inflatable body and the receiver is configured to measure a quantity of energy returned by the inflatable body.

According to another example, the distance sensor 6′ is an ultrasonic sensor.

The ultrasonic sensor comprises a transmitter and a receiver. The transmitter is configured to emit an ultrasonic signal towards the wall of the inflatable body which is returned by the inflatable body. The receiver is configured to count the time elapsed between the emission of the ultrasound signal and its reception to deduce a distance.

According to yet another example, the distance sensor 6′ is a laser sensor. The laser sensor is a «time of flight» sensor comprising a transmitter and a receiver.

The transmitter is configured to emit laser radiation towards the wall of the inflatable body which is returned by the inflatable body. The receiver is configured to count the time elapsed between the emission of the laser radiation and its reception in order to deduce a distance.

In this example the measuring device may comprise a light-emitting diode (LED) intended to provide lighting inside the inflatable body.

In the example illustrated in FIG. 4, said at least one distance sensor is a shooting device such as a camera or a digital camera 6″ and the inflatable body 2 comprises at least one pattern (not shown) or a rib 18 arranged on an inner face of the wall of the inflatable body.

The pattern may for example be a particular random or predetermined color or texture.

The pattern and the rib are intended to allow the shooting device to detect a distortion of the inflatable body.

In the example shown in FIG. 4, the inflatable body 2 comprises a plurality of ribs 18 arranged on the inner face of the wall of the inflatable body 2.

The shooting device makes it possible to know, in real time, a state of the inner face of the wall of the inflatable body. By comparing an image taken at time T1 with an initial image taken at time TO, it is possible to deduce using an image analysis, the increase in the dimension of the inflatable body. For example, the measuring device 6 may comprise a light-emitting diode (LED) 30 intended to provide lighting inside the inflatable body.

In the different examples described above, the distance sensors are connected to the microcontroller 8 of the control device by means of conductive wires 32.

In the examples shown in FIGS. 3 and 4, the conductive wires pass through the handles 10.

The distance sensors make it possible to calculate a radius of the inflatable body.

The diameter is deduced from the calculated radius.

It should be noted that the measuring device can comprise for this purpose a processing unit which can be distinct or shared with the control device

According to a second embodiment illustrated in FIG. 5, the measuring device 6 is arranged outside the inflatable body.

In this embodiment, the inflation device 4 of the inflatable body must comprise the motor 4″ and the solenoid valve 14.

In this embodiment, the system comprises a first part intended to be placed in the genital tract and a second part intended to remain outside the genital tract. The first part comprises the inflatable body 2 and the second part comprises the inflation device 4, the control device 8 and the measuring device 6.

In this embodiment, the measuring device 6 comprises at least one pressure sensor 34 connected to the conduit 12 of the system and at least one computer 40 configured to calculate a quantity of air entering the inflatable body and/or leaving the inflatable body. The pressure sensor 34 is connected to the conduit 12 of the system 1.

For example, the computer 40 is configured to calculate a quantity of air entering the inflatable body using information communicated by the motor 4″.

For example, the computer 40 is configured to calculate a quantity of air leaving the inflatable body by using information communicated by the solenoid valve 14.

The motor 4″, the solenoid valve 14 and the pressure sensor 34 are connected to the control device by means of conductive wires 35, 36, 37. The computer 40 is connected to the control device 8, for example the control device may comprise the computer 40.

The measuring device may comprise a temperature sensor (not shown), connected to the conduit 12 of the system 1.

The measuring device 6 is arranged to determine the dimension of the inflatable body according to a pressure value, a temperature value and a quantity of air in the inflatable body.

It should be noted that the measuring device may comprise for this purpose a processing unit which can be distinct or shared with the control device

In this embodiment, the dimension of the inflatable body is determined based on the ideal gas law and the relationship PV=nRT in which:
P corresponds to the pressure which is determined by means of the pressure sensor 34;
T corresponds to the temperature which is determined either by means of a temperature sensor, in this case the measuring device comprises at least one temperature sensor (not shown), or by an estimate of the ambient temperature: R corresponds to the universal perfect gas constant equal to 8.314 J mol⁻¹ K⁻¹
n corresponds to the quantity of air entering or leaving the inflatable body which is calculated by the computer, and which is determined via information communicated by the motor (the ignition duration of the motor and its flow rate as a function of the pressure makes it possible to know the quantity of air blown by the balloon) and/or via information communicated by the solenoid valve (the opening time of the solenoid valve and the pressure allows knowing the quantity of air leaving the balloon, to be deducted from the quantity of air blown by the motor).

These parameters make it possible to deduce the volume of air present in the inflatable body. The diameter of the inflatable body can be deduced from this volume either by knowing the shape of the inflatable body or empirically by a series of tests making it possible to know the behavior of the inflatable body.

In all the embodiments which have just been described, the system can comprise a gripping means 40 (FIG. 1) intended to allow optimization of the positioning of the inflatable body in the genital tract of the woman and the removal of the inflatable body. For example, the gripping means being a semi-rigid handle.

According to one example, the semi-rigid handle is telescopic. For example, the handle may have a maximum length of 40 cm in the deployed position.

Obviously, the present disclosure is not limited to the examples which have just been described and numerous adjustments can be made to these examples without departing from the scope of the present disclosure. In particular, the different characteristics, shapes, variants and embodiments of the present disclosure can be associated with each other in various combinations to the extent that they are not incompatible or exclusive of each other.

1. A system for preparing a genital tract of a woman before a childbirth, the system comprising:
an inflatable body intended to be placed in the genital tract of the woman, the inflatable body comprising a wall including a first open end intended to allow air to enter the inflatable body or air to leave the inflatable body and a second closed end opposite the first end,
an inflation device of the inflatable body fluidly connected to the first open end of the inflatable body by a conduit,
a measuring device configured to measure a dimension of the inflatable body,
a control device configured to collect measurement data of the dimension measured by the measuring device.

2. The system according to claim 1, wherein the control device is arranged to transmit the measurement data from the measuring device to an interface device configured to receive and display the measurement data by a wireless communication link.

3. The system according to claim 1, wherein the dimension of the inflatable body corresponds to a cross section of the inflatable body.

4. The system according to claim 3, wherein the inflatable body has a substantially circular cross section when the inflatable body is inflated, the system being arranged to determine a diameter of the circular cross section of the inflatable body.

5. The system according to claim 1, wherein the measuring device is integrated into the inflatable body and comprises at least one distance sensor, the inflatable body comprising a central support extending into the inflatable body and the at least one distance sensor being arranged on the central support of the inflatable body, the measuring device being configured to measure a distance between the central support and a point located on an inner face of a wall of the inflatable body.

6. The system according to claim 5, wherein the central support is a semi-rigid rod and the at least one distance sensor is arranged on a free end of the semi-rigid rod.

7. The system according to claim 5, wherein the at least one distance sensor is a sensor selected from the group consisting of an infrared sensor, an ultrasonic sensor and a laser sensor.

8. The system according to claim 5, wherein the at least one distance sensor is a shooting device and the inflatable body comprises at least one pattern or a rib arranged on an inner face of the wall of the inflatable body.

9. The system according to claim 1, wherein the system further comprises a first part intended to be placed in the genital tract and a second part intended to remain outside the genital tract, the first part comprising the inflatable body and the second part comprising the inflation device, the control device and the measuring device, the measuring device comprising at least one pressure sensor, and at least one computer configured to calculate a quantity of air entering the inflatable body and/or a quantity of air leaving the inflatable body.

10. The system according to claim 9, wherein the at least one computer is configured to calculate the quantity of air entering the inflatable body using information communicated by a motor.

11. The system according to claim 9, wherein the at least one computer is configured to calculate the quantity of air leaving the inflatable body using information communicated by a solenoid valve.

12. The system according to claim 1, wherein the second closed end of the inflatable body comprises a bulge.

13. The system according to claim 1, wherein the system comprises a gripping means intended to allow a positioning of the inflatable body in the genital tract of the woman, the gripping means being a semi-rigid handle.

14. The system according to claim 13, wherein the semi-rigid handle is telescopic.

15. The system according to claim 2, wherein the dimension of the inflatable body corresponds to a cross section of the inflatable body.

16. The system according to claim 15, wherein the inflatable body has a substantially circular cross section when the inflatable body is inflated, the system being arranged to determine a diameter of the circular cross section of the inflatable body.

17. The system according to claim 16, wherein the measuring device is integrated into the inflatable body and comprises at least one distance sensor, the inflatable body comprising a central support extending into the inflatable body and the at least one distance sensor being arranged on the central support of the inflatable body, the measuring device being configured to measure a distance between the central support and a point located on an inner face of a wall of the inflatable body.

18. The system according to claim 17, wherein the central support is a semi-rigid rod and the at least one distance sensor is arranged on a free end of the semi-rigid rod.

19. The system according to claim 18, wherein the at least one distance sensor is a sensor selected from the group consisting of an infrared sensor, an ultrasonic sensor and a laser sensor.

20. The system according to claim 6, wherein the at least one distance sensor is a shooting device and the inflatable body comprises at least one pattern or a rib arranged on an inner face of the wall of the inflatable body.
